(19) United States
(12) Patent Application Publication
(10) Pub. No.: US 2019/0355908 A1
(43) Pub. Date: Nov. 21, 2019

(54) Organic Cocrystal and Applications of the Same

(71) Applicants: Wenping HU, Beijing (CN); Yanqiu Sun, Beijing (CN)

(72) Inventors: Wenping HU, Beijing (CN); Yanqiu Sun, Beijing (CN)

(21) Appl. No.: 15/981,912

(22) Filed: May 17, 2018

(51) Int. Cl. H01L 51/00; H01L 51/50; C07C 25/22; C07C 15/38; C07C 255/51

(52) U.S. Cl. CPC H01L 51/0051; H01L 51/0054; H01L 51/5004; C07C 2603/50; C07C 15/38; C07C 255/51; C07C 25/22

(57) ABSTRACT

The present disclosure relates inter alia to an organic cocrystal comprising at least three compounds A, B and C, and the method to prepare the said organic cocrystal, and organic electronic devices comprising the said organic cocrystal, and their application and use.

### BACKGROUND INFORMATION

### 1. Field of the Disclosure

This disclosure relates in general to an organic cocrystal comprising at least 3 components, their use in electronic devices and the electronic devices of the same.

### 2. Description of the Related Art

Organic semiconducting materials have drawn tremendous attention in the past decades, due to their versatility in material design and synthesis, light weight, low cost and solution processibility. Organic electronic devices based on the organic semiconductors, such as organic light-emitting diodes (OLEDs), organic photovoltaics (OPVs) and organic field-effect transistors (OFETs), are becoming the promising next generation technologies in replacement of inorganic counterparts. Actually, OLEDs are becoming commercially successful.

However, organic electronic devices developed to date are mostly based on the amorphous organic films. Because of the amorphous nature, the organic materials are usually worse than the inorganic materials, in some important properties, such as charge carrier mobility, which is required for the high performance devices such as laser and IC. Therefore, organic crystalline materials are of potential importance in the applications.

Organic cocrystals comprising two or more organic molecules provide an efficient and versatile route to construct functional molecular crystals, as reported for example by J. D. Wuest, Nat. Chem. 2012, 4, 74-75; and W. Zhu, et al., J. Am. Chem. Soc. 2015, 137, 11038-11046; In particular, cocrystals comprising 2 components are of particular interest because they commonly exhibit novel or enhanced optoelectronic properties, which are difficult to realize for individual components, as reported for example by S. Horiuchi, et al., Nat. Mater. 2005, 4, 163-166; and G. F. Liu, et al., Angew. Chem. Int. Ed. 2017, 56, 198-202; and S. K. Park, et al., J. Am. Chem. Soc. 2013, 135, 4757-4764; and D. Yan, et al., Angew. Chem. Int. Ed. 2011, 50, 12483-12486; and O. Bolton, et al., Nat. Chem. 2011, 3, 205-210. The performance of organic cocrystals may be tailored by rational design and selection of material combinations, for instance, 2-component CT (Charge-Transfer state) crystals can give rise to an unexpected am bipolar electronic transport performance. Daoben Zhu's group reported fullerene/sulfur-bridged annulene cocrystals with a 2D segregated alternating layer structure were prepared by a simple solution process (J. Zhang, et al., J. Am. Chem. Soc. 2013, 135, 558-561.); and a dual channel p/n organic semiconductors by crystal engineering using hydrogen bonding reported by H. T. Black, D. F. Perepichka, Angew. Chem. Int. Ed. 2014, 53, 2138-2142. But so far, the disclosed organic cocrystals are mostly limited to 2-component cocrystal. Only very few 3-components cocrystals are reported. Liao's group reported TCNB based CT complexes (3-component), which can assemble into a series of luminescent micro tubes that absorb in the visible-light region because of the CT transition from π-conjugated electron donors to the TCNB electron acceptor (Y. L. Lei, et al., Adv. Mater. 2012, 24, 5345-5351; and Y. Q. Sun, et al., Chem. Mater. 2015, 27, 1157-1163.).

Therefore, although a variety of cocrystals, mostly based on CT, have been constructed, they are nonetheless limited to specified complexes, which makes it difficult to achieve the expected functions because of the limited selection of electron D/A pairs and combinations.

There is an urgent need for new organic cocrystals.

### SUMMARY

There is provided an organic cocrystal comprising at least three compounds A, B, and C, wherein A and B are capable of forming a cocrystal and have a type I heterostructure, and B and C are capable of forming a cocrystal and have a type II heterostructure, as described below in the detailed description.

There is also provided an organic crystal, wherein the said compound A or B or C comprises a flat core, which is selected from aromatic group or heteroaromatic group or other flat structures as described below in the detailed description.

There is also provided an organic crystal, wherein the said compound A is selected from at least partially halogenated fused ring system, and the said compound B is selected from non-halogenated fused ring system, as described below in the detailed description.

There is also provided an organic crystal, wherein the said compound A is selected from partially or per fluorinated arene, and/or the said compound B is selected from non-fluorinated arene, as described below in the detailed description.

There is also provided an organic crystal, wherein the said compound C comprises an electron acceptor group, as described below in the detailed description.

There is also provided an organic crystal, wherein the said compound C is equal or less than 20 mol %, as described below in the detailed description.

There is also provided an organic crystal, wherein min (Δ(LUMO_{B}−HOMO_{C}), Δ(LUMO_{C}−HOMO_{B})) is less or equal to the least triplet level of the said compounds B and C, as described below in the detailed description.

There is also provided an organic crystal, wherein the said compounds B and C forms an emissive charge-transfer state (CT) with a maximum wavelength of λ1 in photoluminescence, as described below in the detailed description.

There is also provided an organic crystal, wherein the said CT state has an absorption spectrum, which is at least partially overlapped with the emission spectrum of the said compound B or the cocrystal formed by the said compounds A and B, as described below in the detailed description.

There is also provided a method to prepare the said organic cocrystal as described below in the detailed description.

There is also provided an electronic device comprising the said organic cocrystal as described below in the detailed description.

### BRIEF DESCRIPTION OF DRAWINGS

Embodiments are illustrated in the accompanying figures to improve understanding of concepts as presented herein.

FIG. 1 and FIG. 2 illustrate semiconductor heterojunction structures, showing two possible types of relative positions on energy levels of the highest occupied molecular orbital (HOMO) and the lowest unoccupied molecular orbital (LUMO), when two organic semiconductor materials a and b come into contact.

FIG. 1 includes an illustration of a type-I organic semiconductor heterostructure.

FIG. 2 includes another illustration of a type-II organic semiconductor heterostructure.

FIG. 3 includes illustrations of a) Molecular structures of OFN, pyrene, and TCNB. b) SEM and TEM images of pyrene-OFN microwires, and c, d) FM images of pyrene-OFN and pyrene-TCNB; scale bars 10 μm. e) PL spectra of pyrene-OFN, pyrene, and pyrene-TCNB.

FIG. 4 includes illustrations of XRD patterns of OFN, pyrene and pyrene-OFN crystals deposited on the surface of a quartz substrate.

FIG. 5 includes illustrations of the absorption spectra of a) pyrene-OFN, b) pyrene-TCNB crystals, and their respective constituent materials.

FIG. 6 includes illustrations of a) PL spectra of pyrene-OFN microwires with different TCNB doping ratios (insets: the corresponding photographs excited by UV lamp). The corresponding: b) absorption spectra (dashed lines show the absorption bands of pyrene-TCNB in doped pyrene-OFN microwires); c) time-resolved fluorescence decay; d) FM images of pyrene-OFN microwires; and e) CIE coordinate values. f) Representation of the energy-transfer mechanism.

FIG. 7 includes an illustration of the emission spectrum of pyrene-OFN microwires and absorption spectrum of pyrene-TCNB microtubes.

FIG. 8 includes illustrations of a) Molecular structures of TFP, pyrene, and TCNB. b) FM images of pyrene-TFP microwires upon excitation with 365 nm; TCNB doping concentrations are 0, 0.5, 1, 3, and 100% (from left to right); and d) the corresponding PL spectra. Insets are the corresponding photographs excited by UV lamp. e) Absorption spectra of pyrene-TFP microwires with different TCNB doping ratios. The dashed lines show the absorption bands of pyrene-TCNB in doped pyrene-TFP microwires.

Skilled artisans appreciate that objects in the figures are illustrated for simplicity and clarity and have not necessarily been drawn to scale. For example, the dimensions of some of the objects in the figures may be exaggerated relative to other objects to help to improve understanding of embodiments.

### DETAILED DESCRIPTION

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims. The detailed description addresses the cocrystals comprising at least three components with specific energy structures, the possible compounds and combinations, the method to prepare the said cocrystals, the electronic device comprising the said organic corcrystal, and finally working examples.

The present invention provides an organic cocrystal comprising at least three compounds A, B, and C, wherein A and B are capable of forming a cocrystal and have a type I heterostructure, and B and C are capable of forming a cocrystal and have a type II heterostructure.

In some embodiments, at least one of the said compounds A, B and C is selected from organic compounds;

In some embodiments, at least two of the said compounds A, B and C are selected from organic compounds;

In some embodiments, all of the said compounds A, B and C are selected from organic compounds;

In the context of this disclosure, an organic compound is any chemical compound that contains carbon, except that carbides, carbonates, simple oxides of carbon (for example, CO and CO₂), and cyanides are considered inorganic compounds. That is, the organic compound includes the compounds containing a carbon-hydrogen bond, and the fully halogenated or per fluorinated compounds thereof.

In the embodiments of the present disclosure, the HOMO, LUMO, triplet energy level (T1) and singlet energy level (S1) are playing a key role to the energy level structure of organic materials. Determination to these energy levels is presented as follows.

The energy levels of HOMO and LUMO may be measured by photoelectric effects, including XPS (X-ray photoelectron spectroscopy), UPS (Ultraviolet photoelectron spectroscopy), or CV (Cyclic Voltammetry) method. Recently, a quantum chemical simulation, such as based on the density function theory (DFT), has also become an effective method to calculate the energy structure of the organic compounds.

The triplet energy level T1 of an organic compound may be measured by low temperature time-resolved photoluminescence spectrums, or may be calculated by quantum simulation (such as Time-dependent DFT), for example, by using a commercial software of Gaussian 03W (Gaussian Inc.).

The singlet energy level S1 of an organic compound, may be determined by an absorption spectrum, or a photoluminescence spectrum, may also be calculated by quantum simulation (such as Time-dependent DFT).

A detailed simulation method for HOMO, LUMO, triplet energy level (T1) and singlet energy level (S1) may be referred to WO2011141110 and is also described as bellow.

It should be noted that, an absolute value of HOMO, LUMO, T1 or S1 is each determined by the applied measurement method or calculation method, and even for a same method, different evaluation methods may give different absolute values. For example, different HOMO/LUMO values may be given for a starting point and a peak point in a CV curve. Therefore, a reasonable and meaningful comparison should be made by the same measurement method and the same evaluation method. In the description of the embodiments in the present disclosure, the values of HOMO, LUMO, T1 and S1, are based on a time-dependent DFT simulation, but it does not exclude the applications of other measurement or calculation methods.

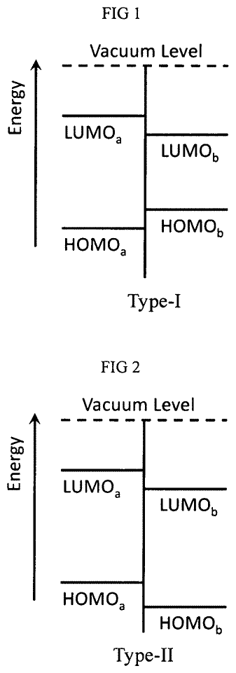

Analogue to the inorganic semiconductors, when two organic semiconductor materials a and b come into contact, they form the so-called heterostructures. Depending the relative positions of HOMO and LUMO, there are two different types of heterostructures, type-I as descripted in FIG. 1, and type-II as descripted in FIG. 2. In the present disclosure, A and B form type-I heterostructure, and B and C form type-II heterostructure. The compounds B and C may form CT state. Compared with the prior art: Y. L. Lei, et al., Adv. Mater. 2012, 24, 5345-5351; and Y. Q. Sun, et al., Chem. Mater. 2015, 27, 1157-1163.), where both A-B, and B-C form CT (thus type-II heterostructure), the present disclosure provides a cocrystal with much wider energy range, because CT state is usually in low-energy.

In one embodiment, the cocrystal according to the present disclosure is a one-dimensional crystal. Preferably, the cocrystal according to the present disclosure is a nanowire.

In another embodiment, the cocrystal according to the present disclosure is a two-dimensional crystal.

In some embodiment, the cocrystal according to the present disclosure is three-dimensional crystal.

In one embodiment, the cocrystal according to the present disclosure can presented by formula AₓB_{y}C_{z}, wherein x, y, z are the molar ratio of the corresponding organic compounds.

In some embodiments, in the said cocrystal AₓB_{y}C_{z}, 0.1≤x≤0.9.

In some embodiments, in the said cocrystal AₓB_{y}C_{z}, 0.1≤y≤0.9.

In some embodiments, in the said cocrystal AₓB_{y}C_{z}, 0<z≤0.4.

In some embodiments, in the said cocrystal AₓB_{y}C_{z}, y=0.5 and x+z=0.5.

In some embodiments, the said cocrystal AₓB_{y}C_{z}, z≤0.3;

In some embodiments, the said cocrystal AₓB_{y}C_{z}, z≤0.2;

In some embodiments, the said cocrystal AₓB_{y}C_{z}, z≤0.1;

In some embodiments, the said cocrystal AₓB_{y}C_{z}, z≤0.05;

In another embodiment, the cocrystal according to the present disclosure comprises further organic compound;

To form cocrystal, A and B, or B and C have to possess a suitable interaction. The suitable interactions for cocrystal formation are for examples, but not limited thereto:

1) Arene-perfluoroarene (AP) interaction: perfluoroarenes are well-known to cocrystallize with arenes or their derivatives in the form of 1:1 molecular complexes, with nearly parallel molecules stacked alternately in the solid state, as reported by C. R. Patrick, G. S. Prosser, Nature 1960, 187, 1021-1021.

2) Charge-Transfer (CT) interaction as reported by Y. L. Lei, et al., Adv. Mater. 2012, 24, 5345-5351; and Y. Q. Sun, et al., Chem. Mater. 2015, 27, 1157-1163.

3) Hydrogen-bonding (HB) interaction as reported by H. T. Black, D. F. Perepichka, Angew. Chem. Int. Ed. 2014, 53, 2138-2142

In a further embodiment, the halogen-bond can be used as the suitable interactions (thereafter “XB interaction”) for cocrystal formation, as reported by Boterashvili et al., in J. Am. Chem. Soc., 2014, 136 (34), pp 11926-11929.

In yet further embodiments, other intermolecular interactions may also be used to form the organic cocrystal.

In some embodiments, in the said cocrystal AₓB_{y}C_{z}, A and B have an AP or HB or XB interaction, and B and C have a CT or HB or XB interaction.

In some embodiments, in the said cocrystal AₓB_{y}C_{z}, A and B have an AP interaction, and B and C have a CT interaction.

In some embodiments, in the said cocrystal AₓB_{y}C_{z}, A and B have an XB interaction, and B and C have a CT interaction.

In some embodiments, in the said cocrystal A_{X}B_{y}C_{Z}, A and C don't have an attractive interaction, or have an attractive interaction less than that of A-B or B-C.

In further embodiments, in the said cocrystal AₓB_{y}C_{z}, A and B or B and C have a mixed interaction with two or more interactions mentioned above.

The organic compounds A, B and C can be selected from specific compounds according to the interaction involved.

In preferred embodiments, A, B and C each has a flat molecular structure or comprises a core structure having a flat molecular geometry. The molecular geometry can be measured by X-ray diffraction or simulated by using a commercial software of Gaussian 03W (Gaussian Inc.).

In some embodiments, in the said cocrystal AₓB_{y}C_{z}, A is selected from at least partially halogenated fused ring system, and B is selected from non-halogenated fused ring system.

In one embodiment, the said fused ring system in compound A or B is a conjugated system.

In some embodiments, in the said cocrystal AₓB_{y}C_{z}, A or B or C comprises a flat core, which is selected from aromatic group or heteroaromatic group.

In this disclosure, an arene or aromatic hydrocarbon or aryl hydrocarbon has the same meaning, and is a hydrocarbon with sigma bonds and delocalized pi electrons between carbon atoms forming a circle. And a heteroarene or heteroaromatic hydrocarbon or heteroaryl hydrocarbon has the same meaning. An aromatic group refers to a hydrocarbyl containing at least one aromatic ring, including monocyclic groups and polycyclic ring systems. Heteroaromatic groups refer to hydrocarbyl groups (containing heteroatoms) that contain at least one heteroaromatic ring, including monocyclic groups and polycyclic ring systems. These polycyclic rings may have two or more rings in which two carbon atoms are shared by two adjacent rings, i.e., a fused ring. At least one of these polycyclic rings is aromatic or heteroaromatic.

Specifically, examples of the aromatic group include benzene, naphthalene, anthracene, phenanthrene, perylene, tetracene, pyrene, benzopyrene, triphenylene, acenaphthene, fluorene, and derivatives thereof.

Specifically, examples of heteroaromatic groups are: furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, o-diazonaphthalene, quinoxaline, phenanthridine, primidine, quinazoline, quinazolinone, and derivatives thereof.

In some embodiments, in the said cocrystal A_{X}B_{y}C_{Z}, A or B comprising a flat core, which is selected from fused aromatic or heteroaromatic groups with 1-10 rings, which is selected from 5-ring or 6-ring.

In some exemplary embodiments, the suitable core for compound A, or B, or C may be further selected from the following general formula as listed in the table 1:

TABLE 1

| | |
|---|---|
| 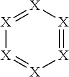 | 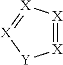 |
| 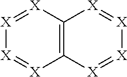 | 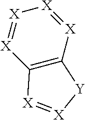 |
| 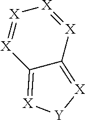 | 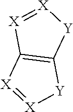 |
| 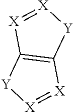 | 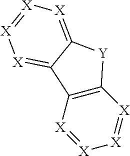 |

wherein X is the same or different in multiple occurrences, independently selected from CR¹ or N;

Y is the same or different in multiple occurrences, independently selected from selected from CR²R³, SiR⁴R⁵, NR⁶ or C(═O), S, or O; and

R¹, R², R³, R⁴, R⁵, and R⁶ in each occurrence are independently selected from H; D; halogen; or a linear alkyl, alkoxy, or thioalkoxy having 1 to 20 C atoms; or a branched or cyclic alkyl, alkoxy or thioalkoxy group, or silyl group having 3 to 20 C atoms; or a substituted ketone group having 1 to 20 C atoms; or an alkoxy group having 2 to 20 C atoms; or an aryloxycarbonyl having 7 to 20 C atoms, cyano (—CN), carbamoyl (—C(═O)NH₂), haloformyl (—C(═O)—X1, wherein X1 represents a halogen atom), formyl (—C(═O)—H), isocyano, isocyanate, thiocyanate, or isothiocyanate, hydroxy, nitro, CF₃, Cl, Br, and F; a crosslinkable group or a substituted or unsubstituted aromatic or heteroaromatic ring system having 5 to 40 annular atoms; or an aryloxy group or heteroaryloxy group having 5 to 40 annular atoms, or a combination thereof, wherein one or more of the groups R¹, R², R³, R⁴, R⁵, and R⁶ may form a monocyclic or polycyclic aliphatic or aromatic ring system with each other and/or with a ring bonded to said groups.

In some exemplary embodiments, R¹, R², R³, R⁴, R⁵, and R⁶ in each occurrence are selected from H; D; halogen; or a linear alkyl, alkoxy, or thioalkoxy having 1 to 10 C atoms; or a branched or cyclic alkyl, alkoxy or thioalkoxy group, or silyl group having 3 to 10 C atoms; or a substituted ketone group having 1 to 20 C atoms; or an alkoxy group having 2 to 10 C atoms; or an aryloxycarbonyl having 7 to 10 C atoms, cyano (—CN), carbamoyl (—C(═O)NH₂), haloformyl (—C(═O)—X1, wherein X1 represents a halogen atom), formyl (—C(═O)—H), isocyano, isocyanate, thiocyanate, or isothiocyanate, hydroxy, nitro, CF₃, Cl, Br, and F; a crosslinkable group or a substituted or unsubstituted aromatic or heteroaromatic ring system having 5 to 20 annular atoms; or an aryloxy group or heteroaryloxy group having 5 to 20 annular atoms, or a combination thereof, wherein one or more of the groups R¹, R², R³, R⁴, R⁵, and R⁶ may form a monocyclic or polycyclic aliphatic or aromatic ring system with each other and/or with a ring bonded to said groups.

In some of the other exemplary embodiments, the suitable cores for compound A, or B, or C is selected from the following structural units in the table 2:

TABLE 2

| | |
|---|---|
| 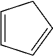 | 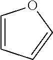 |
| 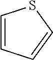 | 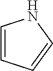 |
| 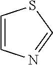 | 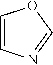 |
| 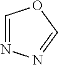 | 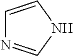 |
| 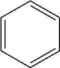 | 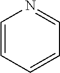 |
| 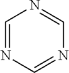 | 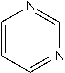 |
| 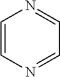 | 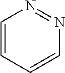 |
| 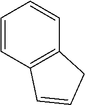 | 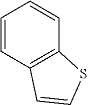 |
| 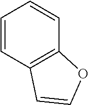 | 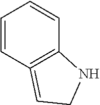 |
| 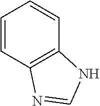 | 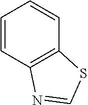 |
| 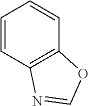 | 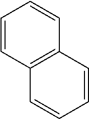 |
| 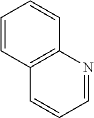 | 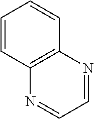 |
| 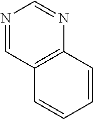 | 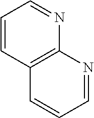 |
| 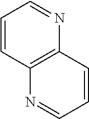 | 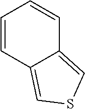 |
| 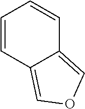 | 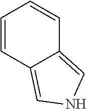 |
| 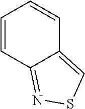 | 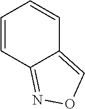 |
| 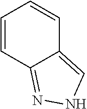 | |

In some embodiments, in the said cocrystal AₓB_{y}C_{z}, C is selected from arene or heteroarene compounds, which is substituted by at least one electron acceptor group.

In some embodiments, in the said cocrystal AₓB_{y}C_{z}, C comprises a core structure as described in table 1 and table 2, which is substituted by at least one electron acceptor group.

Suitable electron acceptor groups may be selected from F, cyano or groups comprising one of the units in the following general formulas as listed in the table 3:

TABLE 3

| | |
|---|---|
| 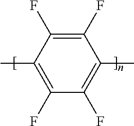 | 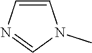 |
| 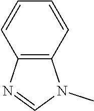 | 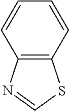 |
| 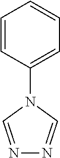 [SMILES: c1ccc(-n2cnnc2)cc1] | 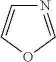 |
| 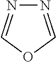 | 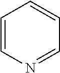 |
| 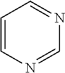 | 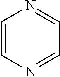 |
| 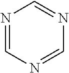 | 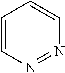 |
| 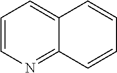 | 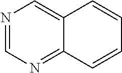 |
| 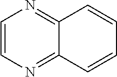 | 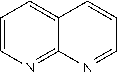 |
| 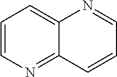 | 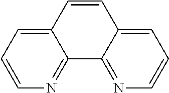 [SMILES: c1cnc2c(c1)ccc1cccnc12] |
| 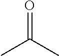 | 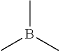 |
| 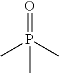 | 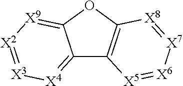 |
| 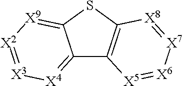 | 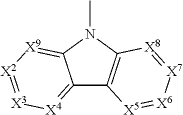 |
| 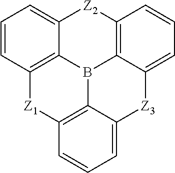 | |

Wherein, n is an integer from 1 to 3; X²-X⁹ are the same or different in multiple occurrences, independently selected from selected from CR or N, and at least one of them is N, Z₁, Z₂, Z₃ in each occurrence are independently selected from N(R), C(R)₂, Si(R)₂, O, C═N(R), C═C(R)₂, P(R), P(═O)R, S, S═O, SO₂ or Null, and R may be selected from the following groups: hydrogen, alkyl, alkoxy, amino, alkene, alkyne, aralkyl, heteroalkyl, aryl and heteroaryl groups.

In an embodiment, the suitable electron acceptor group is selected from the cyano group.

In another embodiment, the suitable electron acceptor group is selected from F.

In one embodiment, in the said cocrystal AₓB_{y}C_{z}, A has a larger band gap than B.

In some embodiments, in the said cocrystal AₓB_{y}C_{z}, A has a smaller conjugated core structure than B.

In another embodiment, in the said cocrystal AₓB_{y}C_{z}, A has a band gap less than or equal to B. The said organic cocrystal may have various electronic or opto-electronic properties.

In one embodiment, the said organic cocrystal have an am bipolar transport property.

In another embodiment, the said organic cocrystal have a light emission property.

In some embodiments, in the said organic cocrystal, compounds B and C forms an emissive charge-transfer state (CT state) with a maximum wavelength of λ1 in photoluminescence.

In some embodiments, in the said organic cocrystal, the said CT state formed by B and C has an absorption spectrum, which is at least partially overlapped with the emission spectrum of B or the cocrystal formed by A and B.

In some embodiments, in the said organic cocrystal, compounds B and C form an exciplex.

In some embodiments, in the said organic cocrystal, min(Δ(LUMO_{B}−HOMO_{C}), Δ(LUMO_{C}−HOMO_{B})) is less or equal to the least triplet level of the said compounds B and C.

In some embodiments, the said organic cocrystal is an emissive crystal.

In one embodiment, the said organic cocrystal is an emissive crystal with an emission wavelength from 380 nm to 1000 nm.

In another embodiment, the said organic cocrystal is an emissive crystal emitting light in red or green or blue.

In yet another embodiment, the said organic cocrystal is an emissive crystal emitting light in broad wavelength range.

In yet another embodiment, the said organic cocrystal is an emissive crystal emitting white light.

In some embodiments, in said the organic cocrystal, the said compounds A, B and C are soluble or dispersible in a same solvent, or soluble or dispersible in the different solvents, which are miscible with each other.

Suitable examples of compound A are set forth in the following table:

| | |
|---|---|
| 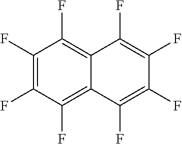 [SMILES: Fc1c(F)c(F)c2c(F)c(F)c(F)c(F)c2c1F] | 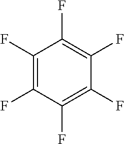 [SMILES: Fc1c(F)c(F)c(F)c(F)c1F] |
| 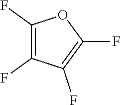 | 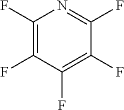 [SMILES: Fc1nc(F)c(F)c(F)c1F] |
| 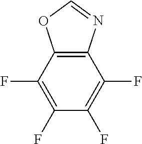 [SMILES: Fc1c(F)c(F)c2ocnc2c1F] | 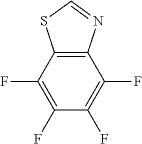 [SMILES: Fc1c(F)c(F)c2scnc2c1F] |
| 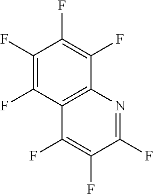 [SMILES: Fc1nc2c(F)c(F)c(F)c(F)c2c(F)c1F] | 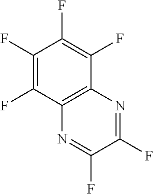 [SMILES: Fc1nc2c(F)c(F)c(F)c(F)c2nc1F] |
| 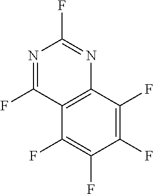 [SMILES: Fc1nc(F)c2c(F)c(F)c(F)c(F)c2n1] | 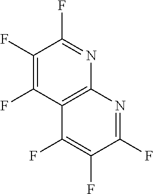 [SMILES: Fc1nc2nc(F)c(F)c(F)c2c(F)c1F] |
| 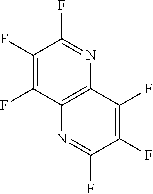 [SMILES: Fc1nc2c(F)c(F)c(F)nc2c(F)c1F] | 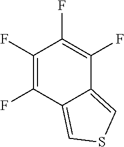 [SMILES: Fc1c(F)c(F)c2cscc2c1F] |
| 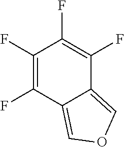 [SMILES: Fc1c(F)c(F)c2cocc2c1F] | 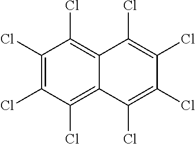 [SMILES: Clc1c(Cl)c(Cl)c2c(Cl)c(Cl)c(Cl)c(Cl)c2c1Cl] |
| 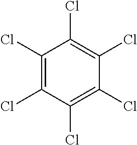 [SMILES: Clc1c(Cl)c(Cl)c(Cl)c(Cl)c1Cl] | 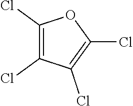 |
| 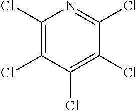 [SMILES: Clc1nc(Cl)c(Cl)c(Cl)c1Cl] | 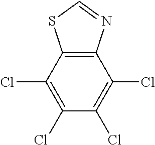 [SMILES: Clc1c(Cl)c(Cl)c2scnc2c1Cl] |
| 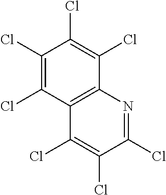 [SMILES: Clc1nc2c(Cl)c(Cl)c(Cl)c(Cl)c2c(Cl)c1Cl] | 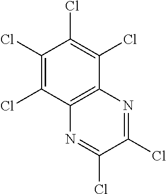 [SMILES: Clc1nc2c(Cl)c(Cl)c(Cl)c(Cl)c2nc1Cl] |
| 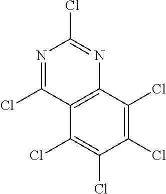 [SMILES: Clc1nc(Cl)c2c(Cl)c(Cl)c(Cl)c(Cl)c2n1] | 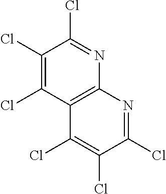 [SMILES: Clc1nc2nc(Cl)c(Cl)c(Cl)c2c(Cl)c1Cl] |
| 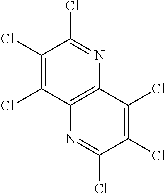 [SMILES: Clc1nc2c(Cl)c(Cl)c(Cl)nc2c(Cl)c1Cl] | 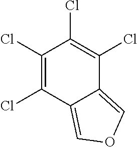 [SMILES: Clc1c(Cl)c(Cl)c2cocc2c1Cl] |
| 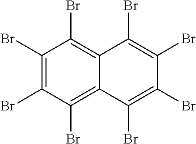 [SMILES: Brc1c(Br)c(Br)c2c(Br)c(Br)c(Br)c(Br)c2c1Br] | 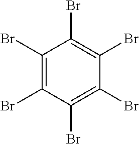 [SMILES: Brc1c(Br)c(Br)c(Br)c(Br)c1Br] |
| 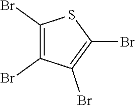 | 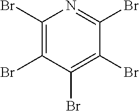 [SMILES: Brc1nc(Br)c(Br)c(Br)c1Br] |
| 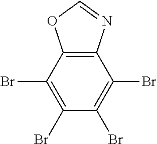 [SMILES: Brc1c(Br)c(Br)c2ocnc2c1Br] | 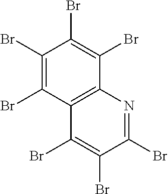 [SMILES: Brc1nc2c(Br)c(Br)c(Br)c(Br)c2c(Br)c1Br] |
| 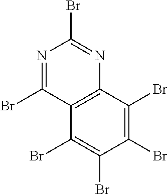 [SMILES: Brc1nc(Br)c2c(Br)c(Br)c(Br)c(Br)c2n1] | 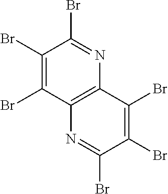 [SMILES: Brc1nc2c(Br)c(Br)c(Br)nc2c(Br)c1Br] |
| 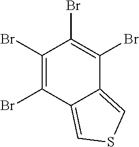 [SMILES: Brc1c(Br)c(Br)c2cscc2c1Br] | 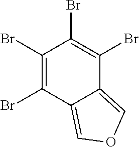 [SMILES: Brc1c(Br)c(Br)c2cocc2c1Br] |
| 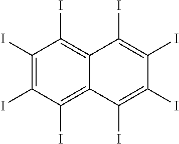 [SMILES: Ic1c(I)c(I)c2c(I)c(I)c(I)c(I)c2c1I] | 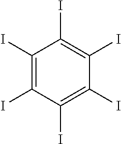 [SMILES: Ic1c(I)c(I)c(I)c(I)c1I] |
| 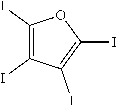 | 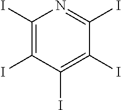 [SMILES: Ic1nc(I)c(I)c(I)c1I] |
| 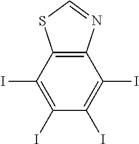 [SMILES: Ic1c(I)c(I)c2scnc2c1I] | 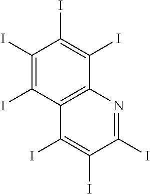 [SMILES: Ic1nc2c(I)c(I)c(I)c(I)c2c(I)c1I] |
| 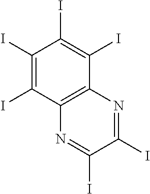 [SMILES: Ic1nc2c(I)c(I)c(I)c(I)c2nc1I] | 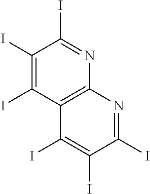 [SMILES: Ic1nc2nc(I)c(I)c(I)c2c(I)c1I] |
| 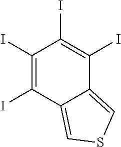 [SMILES: Ic1c(I)c(I)c2cscc2c1I] | 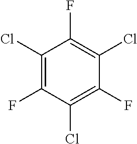 [SMILES: Fc1c(Cl)c(F)c(Cl)c(F)c1Cl] |

Suitable examples of compound B are set forth in the following table:

| | |
|---|---|
| 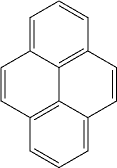 [SMILES: c1cc2ccc3cccc4ccc(c1)c2c34] | 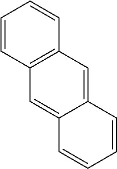 [SMILES: c1ccc2cc3ccccc3cc2c1] |
| 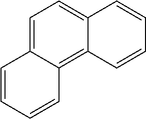 [SMILES: c1ccc2c(c1)ccc1ccccc12] | 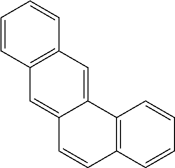 [SMILES: c1ccc2cc3c(ccc4ccccc43)cc2c1] |
| 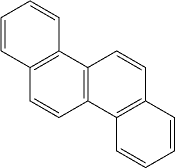 [SMILES: c1ccc2c(c1)ccc1c3ccccc3ccc21] | 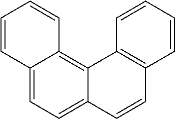 [SMILES: c1ccc2c(c1)ccc1ccc3ccccc3c12] |
| 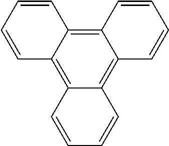 [SMILES: c1ccc2c(c1)c1ccccc1c1ccccc21] | 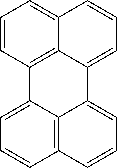 [SMILES: c1cc2cccc3c4cccc5cccc(c(c1)c23)c54] |
| 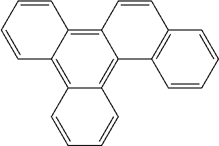 [SMILES: c1ccc2c(c1)ccc1c3ccccc3c3ccccc3c21] | 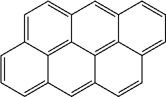 [SMILES: c1cc2ccc3cc4cccc5ccc6cc(c1)c2c3c6c54] |
| 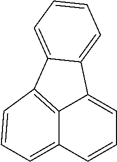 [SMILES: c1ccc2c(c1)-c1cccc3cccc-2c13] | 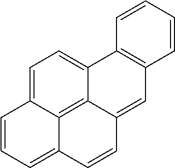 [SMILES: c1ccc2c(c1)cc1ccc3cccc4ccc2c1c34] |
| 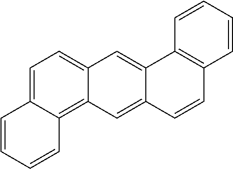 [SMILES: c1ccc2c(c1)ccc1cc3c(ccc4ccccc43)cc12] | 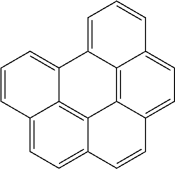 [SMILES: c1cc2ccc3ccc4ccc5cccc6c(c1)c2c3c4c56] |
| 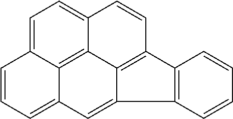 [SMILES: c1ccc2c(c1)-c1ccc3ccc4cccc5cc-2c1c3c45] | 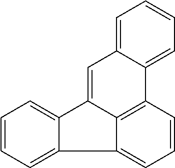 [SMILES: c1ccc2c(c1)-c1cccc3c1c-2cc1ccccc13] |
| 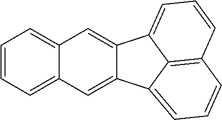 [SMILES: c1ccc2cc3c(cc2c1)-c1cccc2cccc-3c12] | 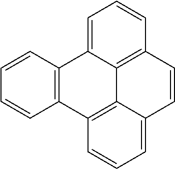 [SMILES: c1ccc2c(c1)c1cccc3ccc4cccc2c4c31] |
| 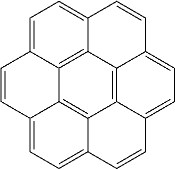 [SMILES: c1cc2ccc3ccc4ccc5ccc6ccc1c1c2c3c4c5c61] | 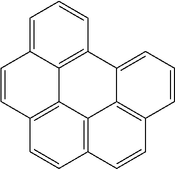 [SMILES: c1cc2ccc3ccc4ccc5cccc6c(c1)c2c3c4c56] |
| 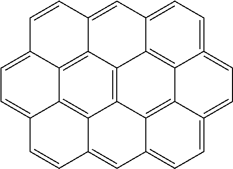 | 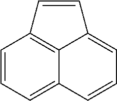 [SMILES: C1=Cc2cccc3cccc1c23] |
| 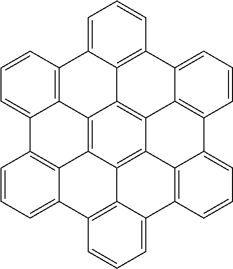 [SMILES: c1cc2c3cccc4c5cccc6c7cccc8c9cccc%10c%11cccc%12c(c1)c2c1c(c34)c(c56)c(c78)c(c%109)c1c%12%11] | 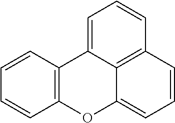 [SMILES: c1ccc2c(c1)Oc1cccc3cccc-2c13] |
| 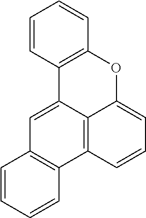 [SMILES: c1ccc2c(c1)Oc1cccc3c1c-2cc1ccccc13] | 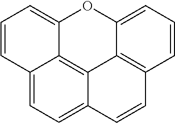 [SMILES: c1cc2c3c(c1)ccc1ccc4cccc(c4c13)O2] |
| 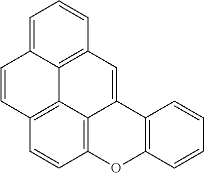 [SMILES: c1ccc2c(c1)Oc1ccc3ccc4cccc5cc-2c1c3c45] | 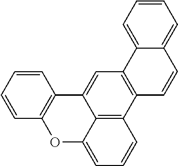 [SMILES: c1ccc2c(c1)Oc1cccc3c1c-2cc1c2ccccc2ccc31] |

Suitable examples of compound C are set forth in the following table:

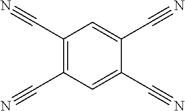 [SMILES: N#Cc1cc(C#N)c(C#N)cc1C#N]

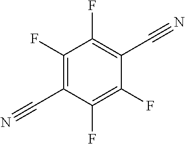 [SMILES: N#Cc1c(F)c(F)c(C#N)c(F)c1F]

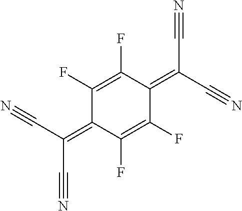 [SMILES: N#CC(C#N)=c1c(F)c(F)c(=C(C#N)C#N)c(F)c1F]

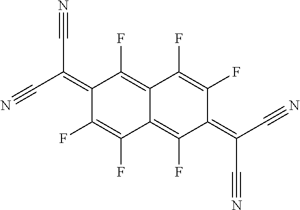 [SMILES: N#CC(C#N)=c1c(F)c(F)c2c(F)c(=C(C#N)C#N)c(F)c(F)c2c1F]

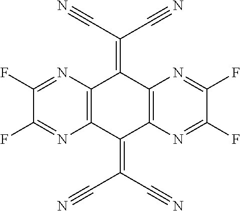 [SMILES: N#CC(C#N)=c1c2nc(F)c(F)nc2c(=C(C#N)C#N)c2nc(F)c(F)nc12]

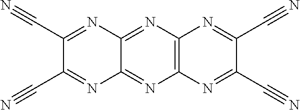 [SMILES: N#Cc1nc2nc3nc(C#N)c(C#N)nc3nc2nc1C#N]

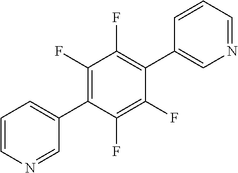 [SMILES: Fc1c(F)c(-c2cccnc2)c(F)c(F)c1-c1cccnc1]

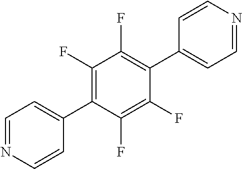 [SMILES: Fc1c(F)c(-c2ccncc2)c(F)c(F)c1-c1ccncc1]

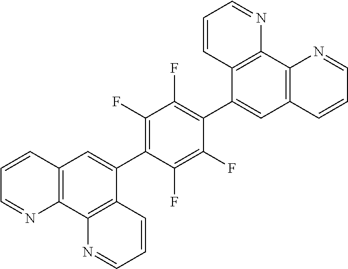 [SMILES: Fc1c(F)c(-c2cc3cccnc3c3ncccc23)c(F)c(F)c1-c1cc2cccnc2c2ncccc12]

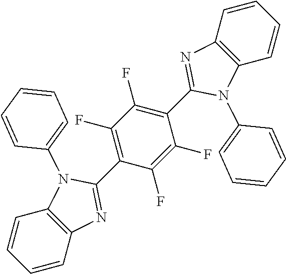 [SMILES: Fc1c(F)c(-c2nc3ccccc3n2-c2ccccc2)c(F)c(F)c1-c1nc2ccccc2n1-c1ccccc1]

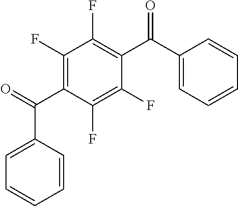 [SMILES: O=C(c1ccccc1)c1c(F)c(F)c(C(=O)c2ccccc2)c(F)c1F]

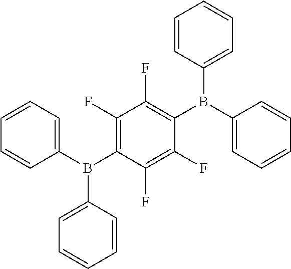 [SMILES: Fc1c(F)c(B(c2ccccc2)c2ccccc2)c(F)c(F)c1B(c1ccccc1)c1ccccc1]

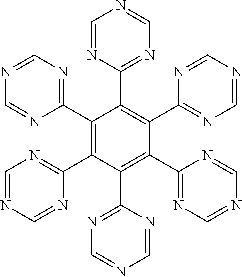 [SMILES: c1ncnc(-c2c(-c3ncncn3)c(-c3ncncn3)c(-c3ncncn3)c(-c3ncncn3)c2-c2ncncn2)n1]

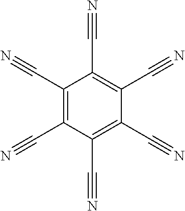 [SMILES: N#Cc1c(C#N)c(C#N)c(C#N)c(C#N)c1C#N]

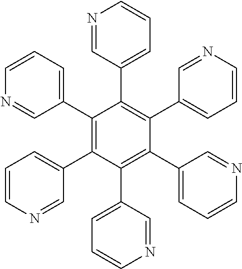 [SMILES: c1cncc(-c2c(-c3cccnc3)c(-c3cccnc3)c(-c3cccnc3)c(-c3cccnc3)c2-c2cccnc2)c1]

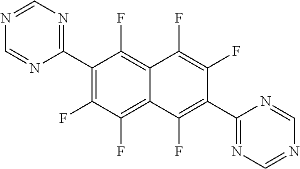 [SMILES: Fc1c(-c2ncncn2)c(F)c2c(F)c(F)c(-c3ncncn3)c(F)c2c1F]

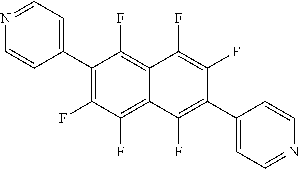 [SMILES: Fc1c(-c2ccncc2)c(F)c2c(F)c(F)c(-c3ccncc3)c(F)c2c1F]

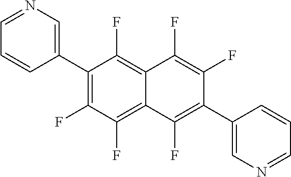 [SMILES: Fc1c(-c2cccnc2)c(F)c2c(F)c(F)c(-c3cccnc3)c(F)c2c1F]

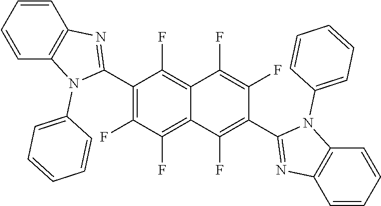 [SMILES: Fc1c(-c2nc3ccccc3n2-c2ccccc2)c(F)c2c(F)c(F)c(-c3nc4ccccc4n3-c3ccccc3)c(F)c2c1F]

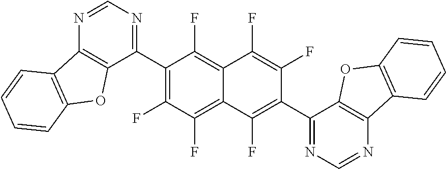 [SMILES: Fc1c(-c2ncnc3c2oc2ccccc23)c(F)c2c(F)c(F)c(-c3ncnc4c3oc3ccccc34)c(F)c2c1F]

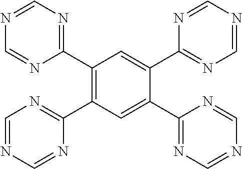 [SMILES: c1ncnc(-c2cc(-c3ncncn3)c(-c3ncncn3)cc2-c2ncncn2)n1]

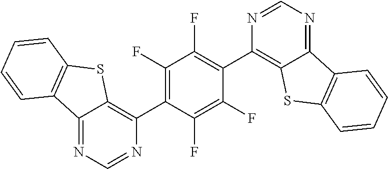 [SMILES: Fc1c(F)c(-c2ncnc3c2sc2ccccc23)c(F)c(F)c1-c1ncnc2c1sc1ccccc12]

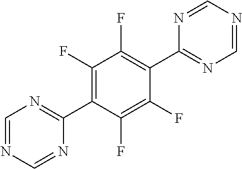 [SMILES: Fc1c(F)c(-c2ncncn2)c(F)c(F)c1-c1ncncn1]

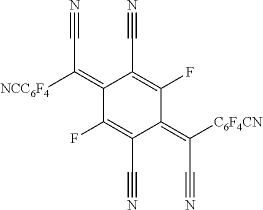

The present disclosure further provides a method to prepare the organic cocrystal as described above and below, comprising:

1) Prepare the solution (or dispersion) 1 of A and C in solvent 1 with a desired ratio;

2) Prepare the solution (or dispersion) 2 of B in solvent 2 wherein solvent 1 and solvent 2 are at least partially miscible;

3) Mix the solution (or dispersion) 1 and solution (or dispersion) 2 with a desired ratio under vigorous stirring or with help of ultrasound;

4) Separate the organic cocrystal from the solution.

The present disclosure further provides another method to prepare the organic cocrystal as described above and below, comprising:

1) Prepare the solution (or dispersion) 1 of A in solvent 1;

2) Prepare the solution (or dispersion) 2 of B and C in solvent 2 with a desired ratio, wherein solvent 1 and solvent 2 are at least partially miscible;

3) Mix the solution (or dispersion) 1 and solution (or dispersion) 2 with a desired ratio under vigorous stirring or with help of ultrasound;

4) Separate the organic cocrystal from the solution (or dispersion).

In a further embodiment, the present disclosure provides a method to prepare the organic cocrystal as described above and below, which comprises a step consisting of physical vapor transport or physical vapor deposition.

The present disclosure yet further provides an electronic device comprising the organic cocrystal as described above and below.

In some embodiments, the said electronic device comprises two electrodes and a functional layer between the said two electrodes, wherein the said functional layer comprises an organic cocrystal according the present disclosure.

In some embodiments, the said electronic device is selected from the group consisting of quantum dot light emitting diodes, quantum dot photovoltaic cells, quantum dot light emitting electrochemical cells, quantum dot field effect transistors, quantum dot light emitting field effect transistors, quantum dot lasers, quantum dot sensors, organic light-emitting diodes, organic photovoltaic cells, organic light-emitting electrochemical cells, organic field effect transistors, organic light-emitting field effect transistors, organic lasers, and organic sensors.

The present disclosure will be described below with reference to following exemplary embodiments, but is not limited thereto. It should be understood that the scope of the present disclosure is defined by the appended claims. Those skilled in the art will appreciate that, guided by the concept of the present disclosure, various modifications can be made to the embodiments of the disclosure, without departing from the spirit and scope of the disclosure as claimed.

### Working Examples

Materials:

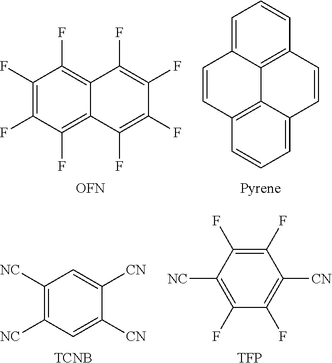

Pyrene (97%), octafluoronaphthalene (OFN, 98%), tetrafluoroterephthalonitrile (TFP, 98%) and 1,2,4,5-tetracyanobezene (TCNB, 97%) were purchased from Sigma-Aldrich. Acetonitrile (HPLC) and ethanol (HPLC) were purchased from Beijing Chemical Ltd. China. All of the chemicals were directly used without further purification. Deionized water (18.2 MΩ·cm⁻¹) was made by a Milli-Q (Millipore) water purification system.

Quantum Chemistry Simulation:

The quantum simulations on organic compounds were conducted in Gaussian 03W (Gaussian Inc.). For organic compound comprising no metal, at first a semi-empirical method “Ground State/Semi-empirical/Default Spin/AM1” (Charge 0/Spin Singlet) was used to optimise the molecular geometry, and then the energy is calculated by TD-DFT (time-dependent density functional theory) method “TD-SCF/DFT/Default Spin/B3PW91” with the basis set “6-31G(d)” (Charge 0/Spin Singlet). For metal complex comprising transition metal (incl. lanthanide and actinide), the geometry optimisation is conducted using Hartree-Fock with Basis Set “LanL2 MB”; and the energy calculation is then conducted by using TD-DFT with correction functional B3PW91 and basis set 6-31G(d) for non-metal elements and Lanz2DZ (Los Alamos National Laboratory 2-double-z) for transition metals. The important results include HOMO/LUMO levels and energies for triplet and singlet excited states. The first triplet and first singlet excited states are most important, and will be called T1 and S1 levels thereafter. From the energy calculation one gets HOMO HEh and LUMO HEh in Hartree units. And the HOMO and LUMO values in electron volts is determined with following equations, which are resulted from the calibration using Cyclovoltametry measurements.

HOMO(eV)=((HEh*27.212)−0.9899)/1.1206

LUMO(eV)=((LEh*27.212)−2.0041)/1.385

These values will be used as HOMO-LUMO levels of the compounds in the present disclosure. As an example, we obtain for the OFN (see also Table 4) from the calculation of a HOMO of −0.23986 Hartree and a LUMO of −0.05271 Hartree, which correspond a calibrated HOMO of −6.71 eV, and a calibrated LUMO of a calibrated −2.85 eV.

TABLE 4

| Compound | HOMO [eV] | LUMO [eV] | S1 [eV] | T1 [eV] |
|---|---|---|---|---|
| OFN | −6.71 | −2.85 | 4.28 | 2.58 |
| Pyrene | −5.75 | −2.60 | 3.75 | 2.07 |
| TCNB | −8.65 | −4.37 | 4.29 | 2.66 |
| TFP | −8.12 | −3.82 | 4.34 | 3.04 |

According to that claculation as shown the the Table 4, OFN and Pyrene from a type-I heterostructure, TCNB and Pyrene from a type-II heterostructure, and TFP and Pyrene from a type-II heterostructure. Using the measured values or other simulation methods as reported in Angew. Chem. Int. Ed. 2017, 56, 1-6, the same conclusion can be drawn. Further TCNB and Pyrene, and TFP and Pyrene meet also the condition: min(Δ(LUMO_{B}−HOMO_{C}), Δ(LUMO_{C}−HOMO_{B})) is less or equal to the least triplet level of the said compounds B and C.

Synthesis of Organic Cocrystal:

The doped pyrene-OFN microwires using TCNB as a dopant (0, 0.5%, 1% and 3%) were synthesized by a liquid-phase self-assembly method. In a typical synthesis, 10 mL of a stock solution of OFN solution containing different TCNB doping concentrations in acetonitrile (COFN=20 mM, CTCNB=0, 0.1, 0.2 and 0.6 mM) was mixed rapidly with an equal volume of pyrene solution in acetonitrile (Cpyrene=20 mM) under vigorous stirring. Meanwhile, the TFP-doped pyrene-OFN and TCNB-doped pyrene-TFP microwires were also prepared by a similar experiment procedure. Besides, pure pyrene-TCNB microtubes were synthesized following the previous experimental method, as reported by Y. L. Lei, et al., Adv. Mater. 2012, 24, 5345-5351. In a typical synthesis, 10 mL of a stock solution of TCNB (CTCNB=20 mM) in acetonitrile was mixed rapidly with an equal volume of pyrene solution (Cpyrene=20 mM) in acetonitrile under vigorous stirring. After several minutes, the resultant solution (2.5 mL) was injected into 10 mL of a 9:11 (v/v) ethanol/water mixture. After several minutes, the flocculent suspension appeared within several seconds. The resultant colloidal samples were collected on the surface of a quartz substrate. Meanwhile, part of the flocculent colloid was separated by centrifugation at 6000 rpm and washed several times with ultrapure water, and finally dried under vacuum for further analysis.

Characterization:

The morphologies and sizes of the samples were examined using field-emission scanning electron microscopy (FESEM, FEI Quanta 200F) at acceleration voltages of 15 kV. Prior to analysis, the samples were coated with a thin gold layer using an Edwards Sputter Coater. TEM images were obtained using a TECNAI T20 electron microscope operated at 80 kV. One drop of the as-prepared colloidal dispersion was deposited on a carbon-coated copper grid, and dried under high vacuum. The X-ray diffraction (XRD) patterns were measured by a D/max 2400 X-ray diffractometer with Cu Kα radiation (λ=1.54050 Å) operated in the 2θ range from 5 to 40°, by using the samples filtered on the surface of a quartz substrate. The photoluminescence (PL) spectra and the solid state fluorescent quantum yield of the samples were measured on a HORIBA JOBTN YVON FLUOROMAX-4 spectrofluorimeter with a slit width of 1 nm. The samples were both deposited on the surface of a quartz substrate. The solid-state absorption spectra of the crystals were conducted on a Cary 5000 UV-VIS-NIR (Varian, USA) and the PL/EX spectra were collected on Horiba FluoroMax-4-NIR spectrophotometers. The absolutely photoluminescence quantum yield (PLQY, Φ) was measured by using an integrating sphere. Inverted fluorescence microscope (Olympus, IX71) equipped with a multispectral imaging system (CRiNuance) with ultraviolet laser (340 nm) was used for testing the microarea PL spectra. The fluorescence lifetime measurements were detected with a streak camera (C5680, Hamamatsu Photonics) dispersed by a polychromator (250is, Chromex) with a spectral resolution of 1 nm and a time resolution of 10 ps. The Commission International de I'Eclairage (CIE) coordinates of the doped pyrene-OFN microwires using TCNB as a dopant were measured by using a PR-670 photometer. Optical absorption spectra of TFP and TCNB were determined used a Jasco V-570 UV-vis spectrophotometer. Cyclic voltammetric measurements were performed in a standard three-electrode cell with a scan rate of 0.1 V s−1 under an inert atmosphere, with Pt/C as the working electrode and Ag as counterelectrode, and Ag/AgCl electrode (saturated KCl) as the reference electrode; n-Bu4NPF6 (0.1 M) was used as supporting electrolyte and Fc/Fc+ was used as an internal standard.

Results:

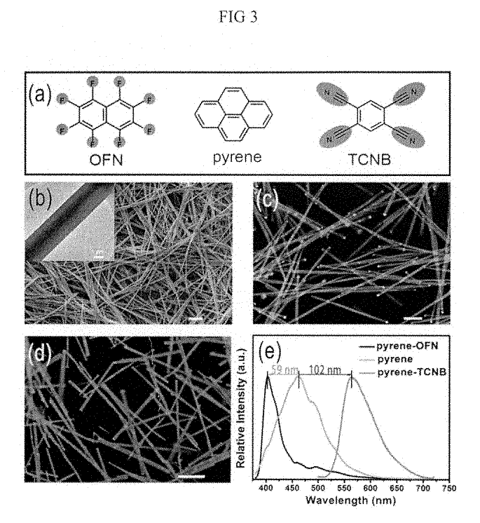

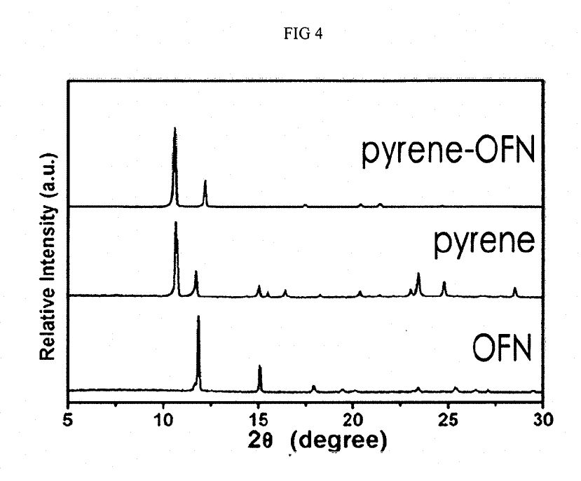

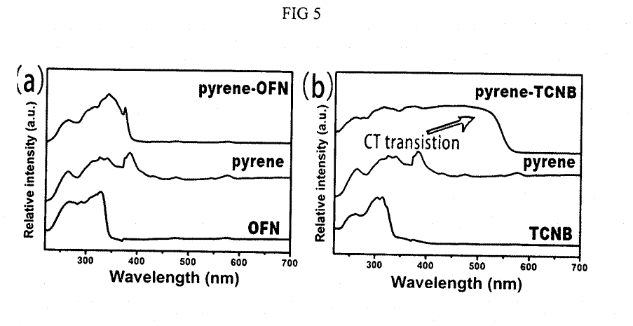

One-dimensional (1D) solid wires formed instantly upon rapid mixing of pyrene and OFN in acetonitrile, as demonstrated by scanning electron (SEM) and transmission electron (TEM) microscopy images shown in FIG. 3b. Obviously, the X-ray diffraction (XRD) pattern of the cocrystal is distinct from that of each constituent molecule (as shown in FIG. 4), further suggesting the formation of a pyrene-OFN cocrystal. The fluorescence microscopy (FM) image of the pyrene-OFN microwires (FIG. 3c) reveals that they emit uniform and strong blue light, in contrast to pure pyrene or OFN. Hence, a luminescent pyrene-OFN cocrystal can be realized readily through arene-perfluoroarene interactions. A typical CT complex, pyrene-TCNB, was selected as a model material for comparison and the resultant microtubes emitted orange light (FIG. 3d).

Photoluminescence (PL) spectroscopy was performed on the two types of pyrene-based cocrystals to investigate their respective luminescence properties. As shown in FIG. 3e, the PL spectrum from pure pyrene crystals presents a main emission peak at around 462 nm, which might be due to excimer formation. In contrast, pyrene-OFN displays a strong PL band at 403 nm, as well as weak peaks in the range of 450-525 nm, whereas pyrene-TCNB has a single band at 564 nm. Interestingly, when compared to the PL spectrum of pure pyrene the former shows a blue-shift at about 59 nm and the latter presents a red-shift up to 102 nm.

As described before (Y. L. Lei, et al., Adv. Mater. 2012, 24, 5345-5351, and C. R. Patrick, G. S. Prosser, Nature 1960, 187, 1021-1021.), the absorption spectra can be used to distinguish CT and AP interactions because the latter does not exhibit characteristic CT absorption bands in UV spectra. The different photophysical natures of pyrene-OFN and pyrene-TCNB are clearly exhibited in FIGS. 5a and 5b, where the absorption spectra of pyrene-OFN, pyrene-TCNB, and their respective constituent materials are shown. Typically, the absorption bands of pyrene-OFN can almost be regarded as the sum of the absorption bands of pure pyrene and OFN, without an obvious red-shift. Pyrene-TCNB exhibits a broader red-shifted peak at around 510 nm (corresponding to the CT transition from pyrene to TCNB) compared to the constituent molecules.

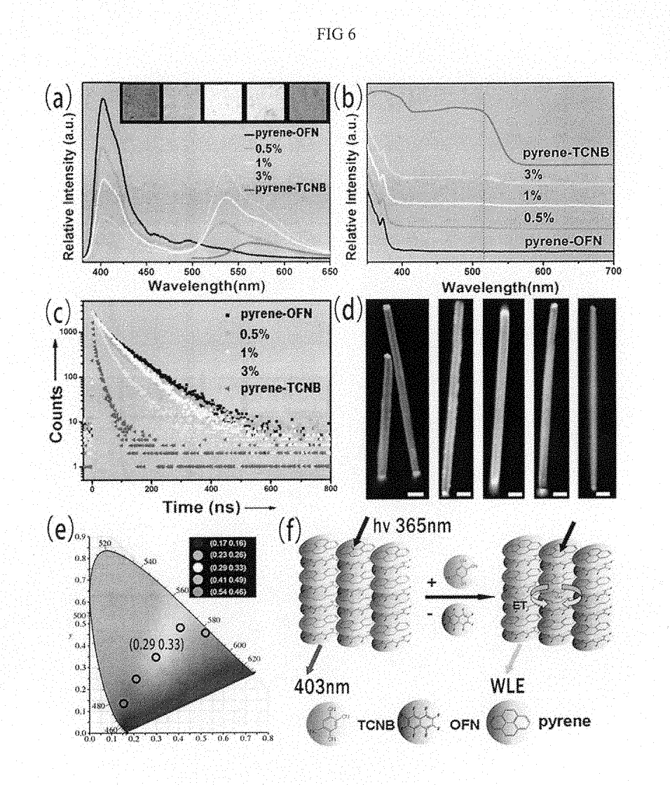

Upon excitation with a UV lamp, the TCNB-doped pyrene-OFN microwires with different doping ratios exhibited tunable emission colors involving white-light emission (WLE) at a 1% doping ratio (inset, FIG. 6 a). Besides the peak at 403 nm arising from the pyrene-OFN component, the corresponding PL spectrum also reveals two splitting peaks at 535 and 575 nm resulting from the pyrene-TCNB system at a doping ratio of 0.5% (green curve, FIG. 6 a). Emission bands derived from the former would reduce gradually, whereas those originating from the latter would increase upon inflation of the doping ratios to 1% (white curve) and 3% (yellow curve). At low doping ratios (0.5% and 1%), the corresponding absorption spectra shown in FIG. 6 b are almost unchanged relative to that of pure pyrene-OFN, while an additional weak absorption peak at around 515 nm was detected at a 3% doping ratio, which is clearly derived from pure pyrene-TCNB.

Moreover, the pure pyrene-OFN microwires have a fluorescence lifetime of about 66.2 ns, which would reduce to 32.3 ns at a 3% doping ratio (FIG. 6 c). Thus, an energy-transfer process from pyrene-OFN to pyrene-TCNB can occur. FM observation results reveal that the doped pyrene-OFN wires display uniform and tunable emission colors ranging from blue to yellow when increasing the doping ratios from 0.5% to 3% (FIG. 6 d). Thereby, it can be deduced that pyrene-TCNB molecules were uniformly doped into the pyrene-OFN host, which was also confirmed by XRD (Supporting Information, FIG. S7). Furthermore, the CIE color coordinate values of pyrene-OFN microwires with WLE are (0.29, 0.33), which is close to ideal white light (FIG. 6 e).

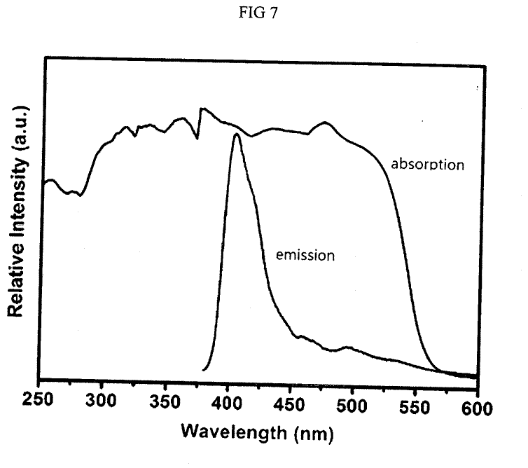

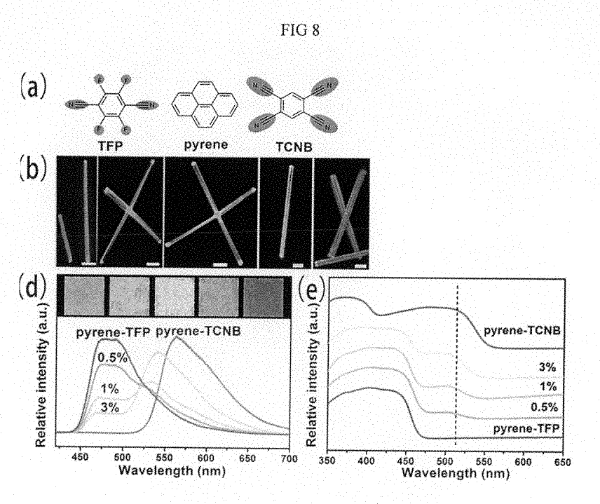

A possible energy-transfer mechanism for the doped pyrene-OFN microwires is displayed in FIG. 6 f. Firstly, pyrene-OFN/pyrene-TCNB D/A pairs fulfill the well-matched structural requirement ascertained from the structural analysis. Secondly, a good overlap of the emission spectrum of pyrene-OFN and the absorption spectrum of pyrene-TCNB (FIG. 7) is also satisfied. Hence, it can be expected that an efficient Förster resonance energy transfer (FRET) process from pyrene-OFN to pyrene-TCNB could occur. Specifically, once pyrene-OFN donor is excited by 365 nm light its excitation energy will transfer to a nearby pyrene-TCNB acceptor located within a distance of 2-6 nm. Moreover, specific ordered supramolecular stacking modes derived from CT and AP interactions will also facilitate the occurrence of efficient energy transfer. Importantly, competitive intermolecular interactions (including CT and AP interactions in the present system) enable a doping process because the former has a larger associative ability relative to the latter, as reported by M. D. Gujrati, et al., Langmuir 2011, 27, 6554-6558.

Another electron acceptor, tetrafluoroterephthalonitrile (TFP; FIG. 8 a) was also applied to form a two-component cocrystal with a pyrene donor, that is, pyrene-TFP. Considering that TFP contains four fluorine atoms and two-CN groups, it can be expected that AP and CT interactions may be both involved in the pyrene-TFP system. As a result, the pyrene-TFP microwires emit blue light upon excitation by UV light (FIG. 8 b). The FM results shown in FIG. 8 b indicate that pure pyrene-TFP microwires emit strong blue light, whereas TCNB-doped pyrene-TFP microwires with doping ratios ranging from 0.5% to 3% show tunable emission colors from blue-green to yellow, which are consistent with their corresponding photographs (inset of FIG. 8 d). Moreover, the PL (FIG. 8 d) and absorption (FIG. 8 e) spectra of the doped pyrene-TFP microwires were also examined to ascertain the occurrence of an efficient energy-transfer process from pyrene-TFP to pyrene-TCNB.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

1. An organic cocrystal comprising at least three compounds A, B, and C, wherein A and B are capable of forming a cocrystal and have a type I heterostructure, and B and C are capable of forming a cocrystal and have a type II heterostructure.

2. The organic cocrystal of claim 1, wherein the compound A or B or C comprises a flat core, and a flat core is selected from aromatic group or heteroaromatic group.

3. The organic cocrystal of claim 1, wherein the compound A or B or C comprises a flat core, which is selected from the structures represented by the following general formula: 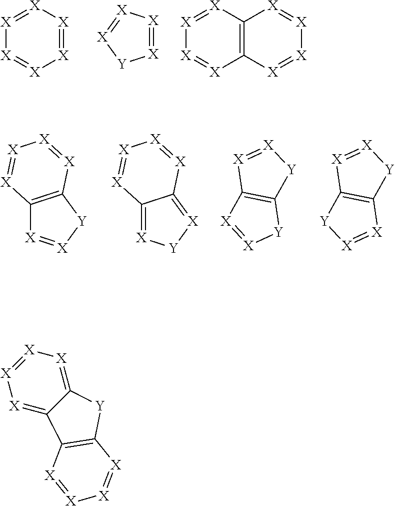
wherein X is the same or different in multiple occurrences, independently selected from CR′ or N;
Y is the same or different in multiple occurrences, independently selected from selected from CR²R³, SiR⁴R⁵, NR⁶ or C(═O), S, or O; and
R₁, R₂, R₃, R₄, R₅, and R₆ in each occurrence are independently selected from H; D; halogen;
or a linear alkyl, alkoxy, or thioalkoxy having 1 to 20 C atoms; or a branched or cyclic alkyl, alkoxy or thioalkoxy group, or silyl group having 3 to 20 C atoms; or a substituted ketone group having 1 to 20 C atoms; or an alkoxy group having 2 to 20 C atoms; or an aryloxycarbonyl having 7 to 20 C atoms, cyano (—CN), carbamoyl (—C(═O)NH₂), haloformyl (—C(═O)—X1, wherein X1 represents a halogen atom), formyl (—C(═O)—H), isocyano, isocyanate, thiocyanate, or isothiocyanate, hydroxy, nitro, CF₃, Cl, Br, and F; a crosslinkable group or a substituted or unsubstituted aromatic or heteroaromatic ring system having 5 to 40 annular atoms; or an aryloxy group or heteroaryloxy group having 5 to 40 annular atoms, or a combination thereof, wherein one or more of the groups R₁, R₂, R₃, R₄, R₅, and R₆ may form a monocyclic or polycyclic aliphatic or aromatic ring system with each other and/or with a ring bonded to said groups.

4. The organic cocrystal of claim 1, wherein the compound A is selected from at least partially halogenated fused ring system, and the compound B is selected from non-halogenated fused ring system.

5. The organic cocrystal of claim 4, wherein the fused ring system is a π-conjugated system.

6. The organic cocrystal of claim 1, wherein the compound A is selected from partially or per fluorinated arene.

7. The organic cocrystal of claim 1, wherein the compound B is selected from no fluorinated arene.

8. The organic cocrystal of claim 1, wherein the compound A has a larger band gap than the compound B

9. The organic cocrystal of claim 1, wherein the compound A is selected from the following groups:

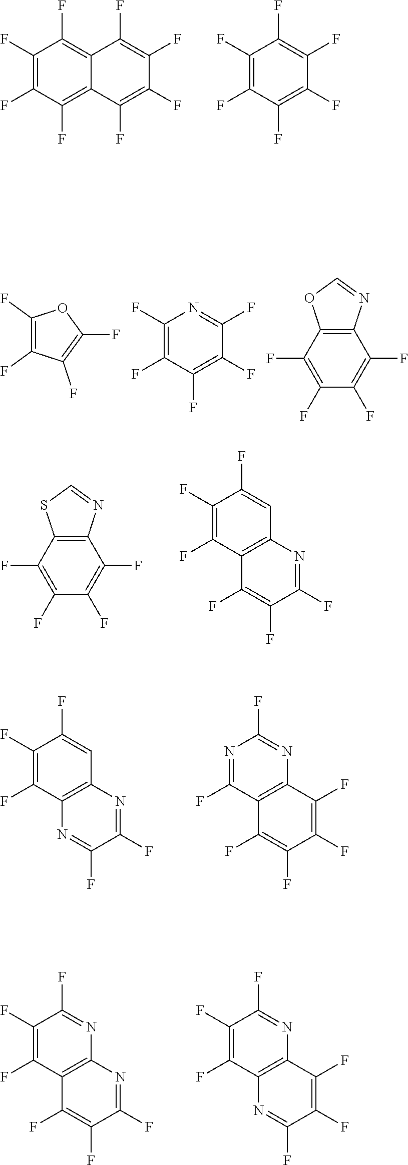

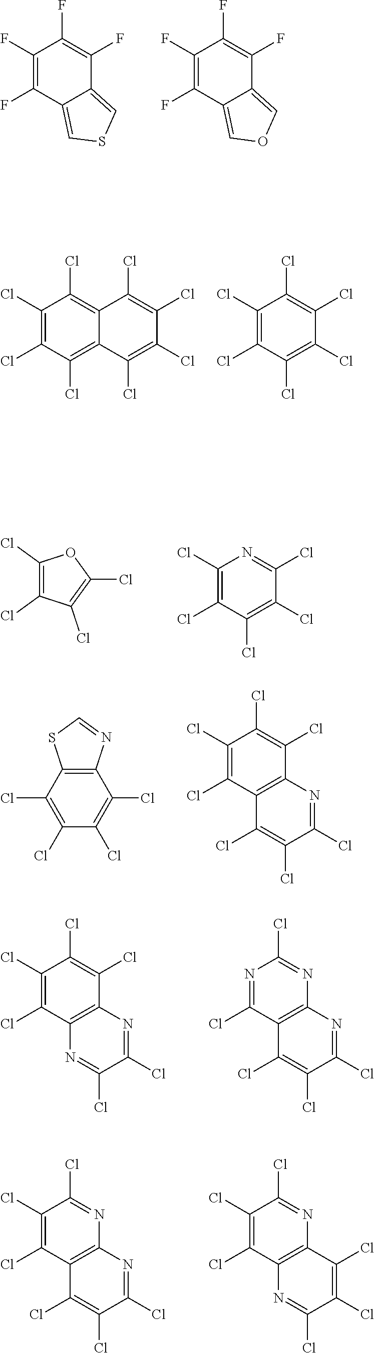

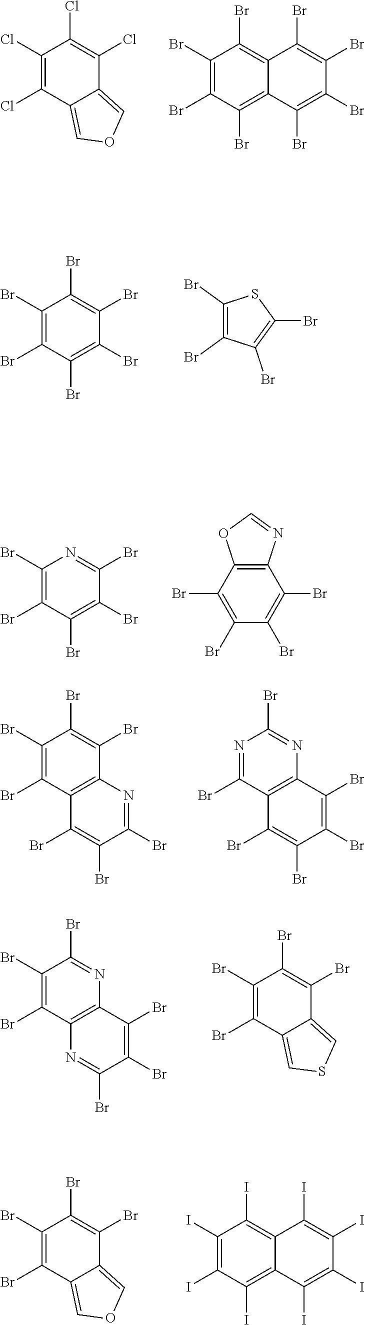

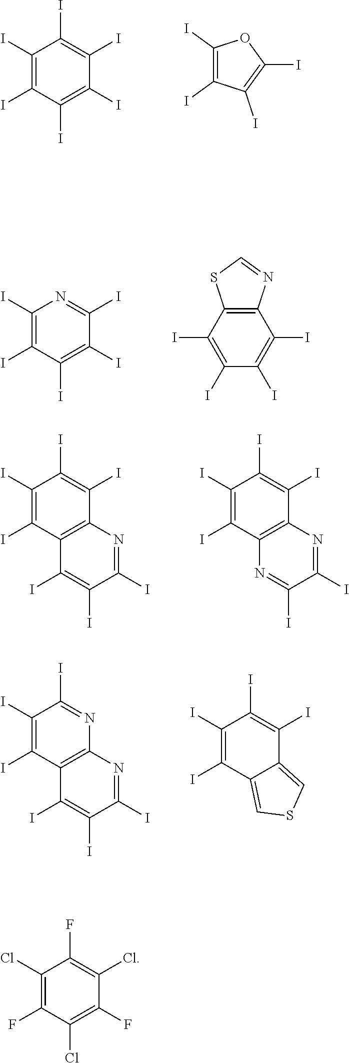

10. The organic cocrystal of claim 1, wherein the compound B is selected from:

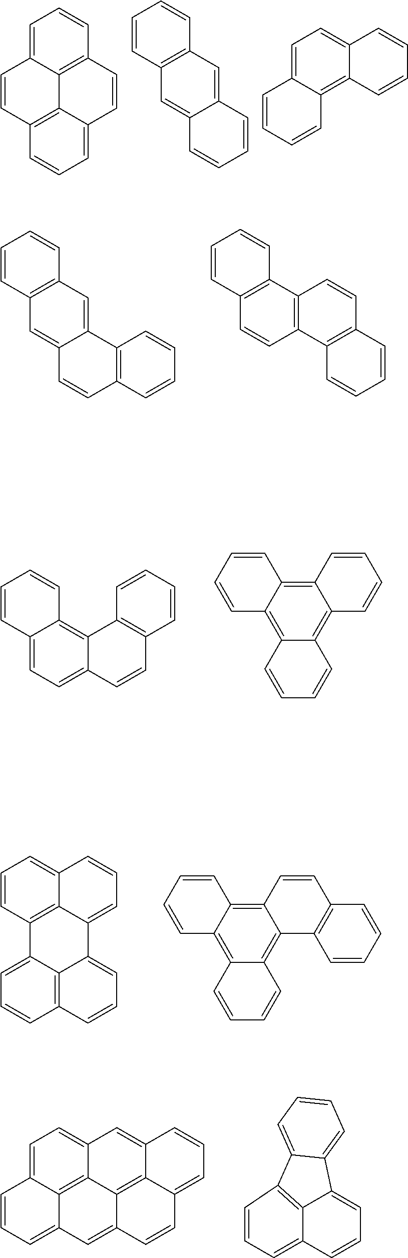

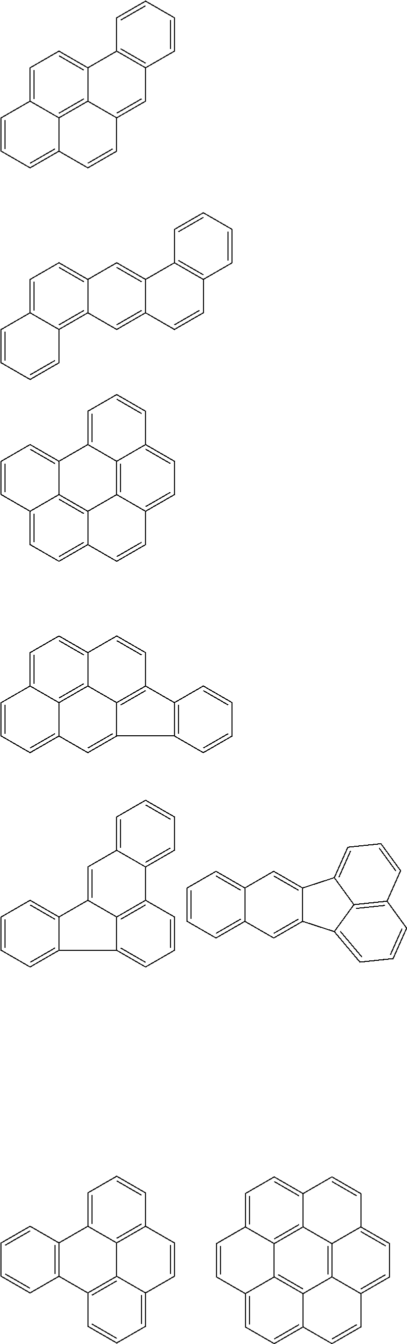

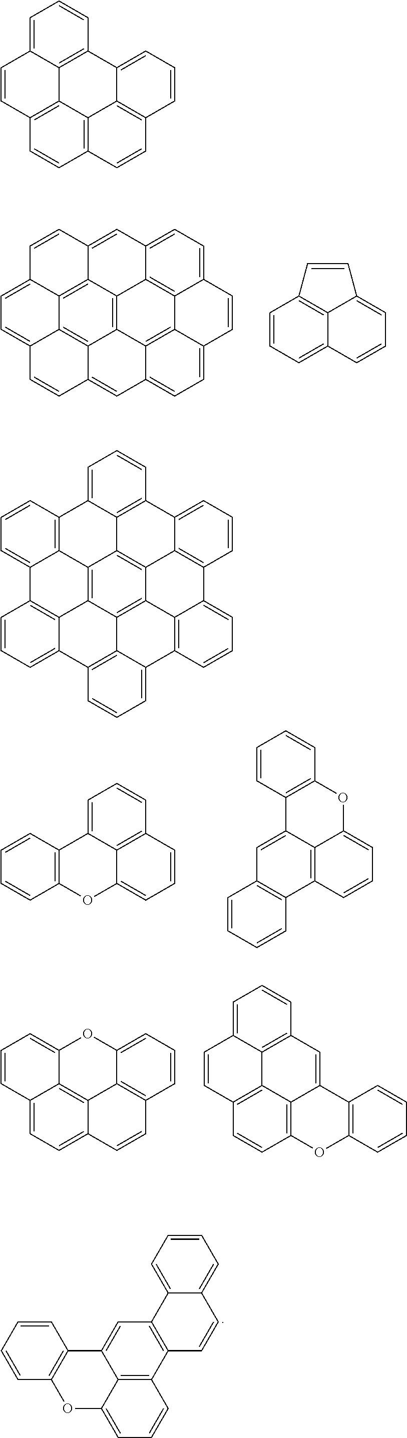

11. The organic cocrystal of claim 1, wherein the compound C comprising an electron acceptor group selected from F, cyano or groups comprising one of the units in the following general formulas: 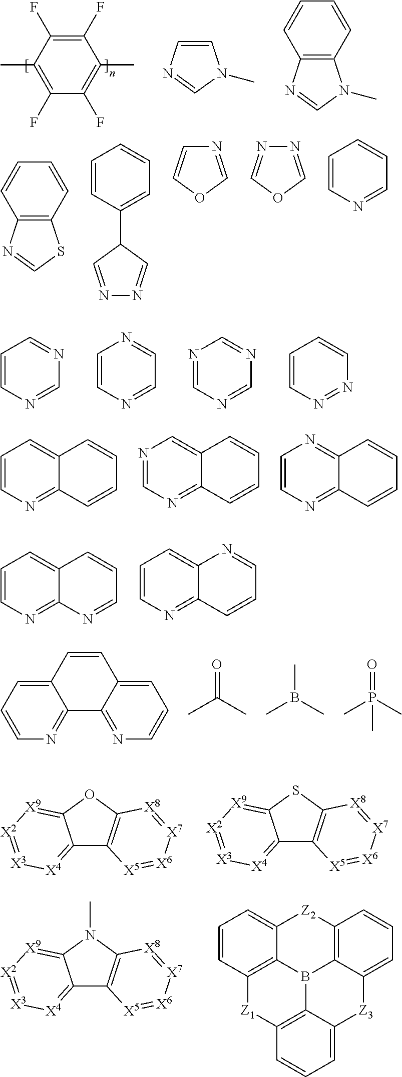
Wherein, n is an integer from 1 to 3; X²-X⁹ are the same or different in multiple occurrences, independently selected from selected from CR or N, and at least one of X²-X⁹ is N, Z₁, Z₂, Z3 in each occurrence are independently selected from N(R), C(R)₂, Si(R)₂, O, C═N(R), C═C(R)₂, P(R), P(═O)R, S, S═O, SO₂ or Null, and R is selected from the group consisting of: hydrogen, alkyl, alkoxy, amino, alkene, alkyne, aralkyl, heteroalkyl, aryl and heteroaryl groups

12. The organic cocrystal of claim 1, wherein the compound C is selected from:

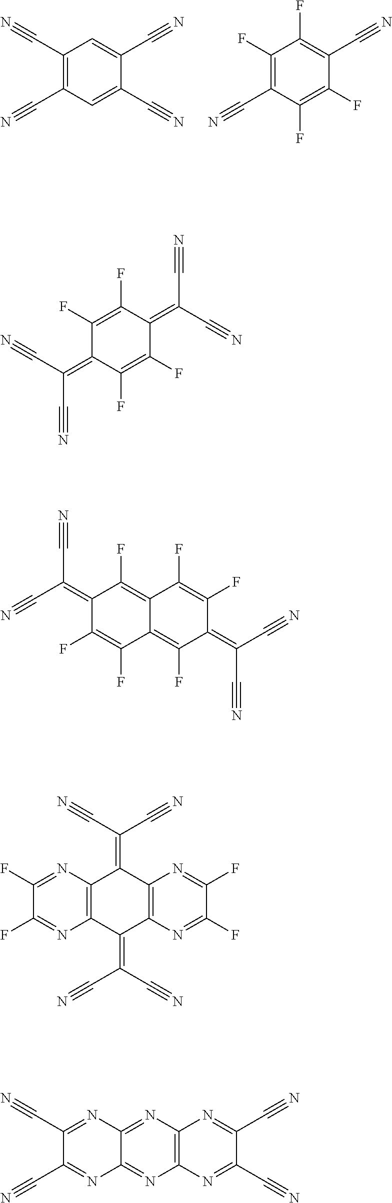

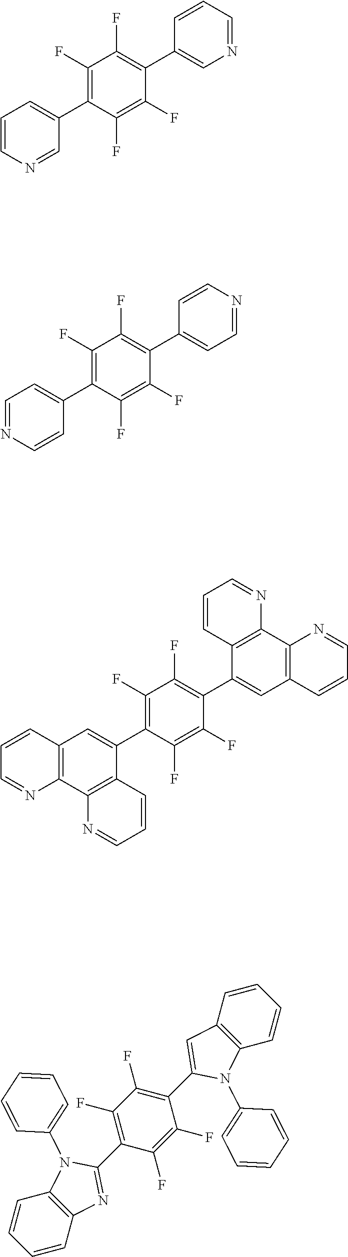 [SMILES: Fc1c(F)c(-c2cccnc2)c(F)c(F)c1-c1cccnc1 ; Fc1c(F)c(-c2ccncc2)c(F)c(F)c1-c1ccncc1 ; Fc1c(F)c(-c2cc3cccnc3c3ncccc23)c(F)c(F)c1-c1cc2cccnc2c2ncccc12 ; Fc1c(F)c(-c2nc3ccccc3n2-c2ccccc2)c(F)c(F)c1-c1cc2ccccc2n1-c1ccccc1]

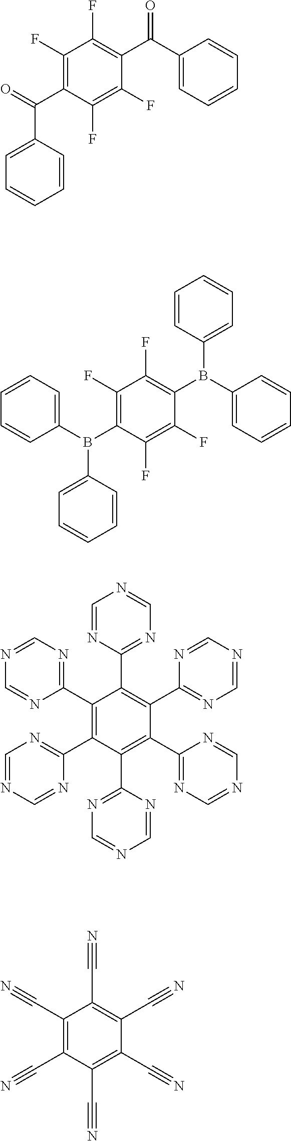 [SMILES: O=C(c1ccccc1)c1c(F)c(F)c(C(=O)c2ccccc2)c(F)c1F ; Fc1c(F)c(B(c2ccccc2)c2ccccc2)c(F)c(F)c1B(c1ccccc1)c1ccccc1 ; c1ncnc(-c2c(-c3ncncn3)c(-c3ncncn3)c(-c3ncncn3)c(-c3ncncn3)c2-c2ncncn2)n1 ; N#Cc1c(C#N)c(C#N)c(C#N)c(C#N)c1C#N]

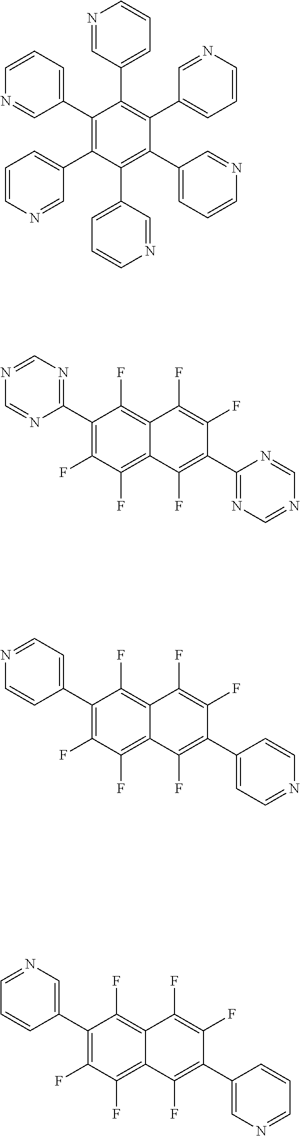 [SMILES: c1cncc(-c2c(-c3cccnc3)c(-c3cccnc3)c(-c3cccnc3)c(-c3cccnc3)c2-c2cccnc2)c1 ; Fc1c(-c2ncncn2)c(F)c2c(F)c(F)c(-c3ncncn3)c(F)c2c1F ; Fc1c(-c2ccncc2)c(F)c2c(F)c(F)c(-c3ccncc3)c(F)c2c1F ; Fc1c(-c2cccnc2)c(F)c2c(F)c(F)c(-c3cccnc3)c(F)c2c1F]

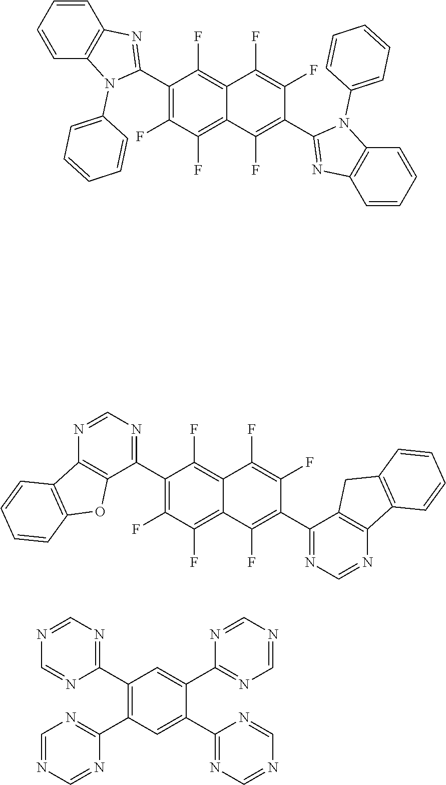 [SMILES: Fc1c(-c2nc3ccccc3n2-c2ccccc2)c(F)c2c(F)c(F)c(-c3nc4ccccc4n3-c3ccccc3)c(F)c2c1F ; Fc1c(-c2ncnc3c2oc2ccccc23)c(F)c2c(F)c(F)c(-c3ncnc4c3Cc3ccccc3-4)c(F)c2c1F ; c1ncnc(-c2cc(-c3ncncn3)c(-c3ncncn3)cc2-c2ncncn2)n1]

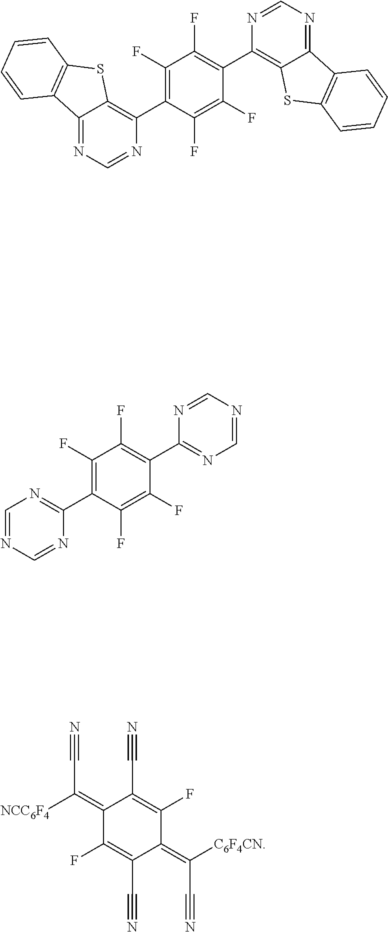

13. The organic cocrystal of claim 1, wherein the compound C is equal or less than 20 mol %.

14. The organic cocrystal of claim 1, wherein min(Δ(LUMO_{B}−HOMO_{C}), Δ(LUMO_{C}−HOMO_{B})) is less or equal to the least triplet level of the compounds B and C.

15. The organic cocrystal of claim 1, wherein the compounds B and C forms an emissive charge-transfer state (CT) with a maximum wavelength of λ1 in photoluminescence

16. The organic cocrystal of claim 12, wherein the CT state has an absorption spectrum, which is at least partially overlapped with the emission spectrum of B or the cocrystal formed by the compounds A and B.

17. The organic cocrystal of claim 1, wherein the compounds A, B and C are soluble in a same solvent.

18. The organic cocrystal of claim 1 is a nanowire.

19. A method to prepare the organic cocrystal of claim 1 comprising the following steps:
1) Prepare the solution 1 of compounds A and C in solvent 1 with a desired ratio;
2) Prepare the solution 2 of compound B in solvent 2 wherein solvent 1 and solvent 2 are at least partially miscible;
3) Mix the solution 1 and solution 2 with a desired ratio under vigorous stirring or with help of ultrasound;
4) Separate the organic cocrystal from the solution.
or comprising the following steps:
1) Prepare the solution 1 of A in solvent 1;
2) Prepare the solution 2 of B and C in solvent 2 with a desired ratio, wherein solvent 1 and solvent 2 are at least partially miscible;
3) Mix the solution 1 and solution 2 with a desired ratio under vigorous stirring or with help of ultrasound;
4) Separate the organic cocrystal from the solution.

20. An opto-electronic device comprising an organic cocrystal of claim 1.
